SEQUENCE LISTING (3 sequences)

SEQ ID NO: 1 - protein, 460 aa, Unknown
  Sequence obtained from environmental sample
ATGFYVNGNTLYDATGSPFVMRGINHAHSWFKDDSSTAIPAIAKTGANTIRIVLSDGSQYTKDDINTVKSLISLAEKNNLIAILEVHDATGNDAVSSLNDAVSYWISIKEALIGKEDRVLINIANEWYGTWDGASWASGYKQAIPKLRDAGLSHTLIVDSAGWGQYPESIHQYGKDVFNADPLKNTMFSIHMYEYAGGDASTIKSNIDGVLNQDLALIIGEFGHKHTNGDVDEETIMSYSQQKNVGWLAWSWKGNGPEWSYLDLSNDWAGDNLTSWGNTIVNGANGLKATSKISPVFDGGDHPGGSGGTENTLYNFETETQSWSGGNVMAGPWSTNEWASKDNYSLKADVQLNNNSQHYLSLTQNQNFSGKSQLKATVKHADWGNLGNGINAQLYVKTGSDWKWFDGESVEINSSNGTILTLDLSSISDLNDIKEIGVQFMGSSKSSGQTAVYVDNVTIQ

SEQ ID NO: 2 - protein, 297 aa, Artificial Sequence
  Synthetic
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTV

SEQ ID NO: 3 - protein, 466 aa, Unknown
  Bacillus galactosidilyticus
ASGFYVNGNTLYDATGTPFVIRGINHAHSWFKDDTATAIPAIAATGANTIRIVLSDGSQYSRDDIDGVRNLISLAEENNLIAMLEVHDATGKDDISSLDSVADYWISIKEALIGKEDKVLINIANEWYGTWDGASWADGYKQVIPKLRNAGLNHTLIVDSAGWGQFPESIHNYGKEVFNADPLQNTMFSIHMYEYAGGDASTVKANIDGVLNQGLAVIIGEFGHRHTDGDVDEATIMNYSQEKNVGWLAWSWKGNGMEWDYLDLSYDWAGNNLTDWGNTIVNSTNGLKATSEISPVFGDGDDGVGDGGPGDSNGTETTLYNFETGTEGWSGENIETGPWSVNEWAAKGNHSLKADVNLGDNSEHYLYLTQNLNFSGKSQLTATVKHADWGNFGDEINAKLYVKTGSDWQWFDGGIEKINSSIGTIITLDLSSLSNPSDIKEVGVQFTGSSNSYGLTALFVDNVTIK

(19) United States
(12) Patent Application Publication
(10) Pub. No.: US 2023/0141942 A1
(43) Pub. Date: May 11, 2023

(54) MANNANASE FOR FORMULATIONS HAVING PH 5-12

(71) Applicant: BASF SE, Ludwigshafen (DE)

(72) Inventors: Amy Migliori, San Diego, CA (US); Asfia Qureshi, San Diego, CA (US); Amanda Rae Logue, San Diego, CA (US); Zachary David Miles, San Diego, CA (US); Cindy Hoang, San Diego, CA (US); Jesper Nielsen, San Diego, CA (US)

(21) Appl. No.: 17/762,570

(22) PCT Filed: Sep. 22, 2020
(86) PCT No.: PCT/EP2020/076359

§ 371 Date: Mar. 22, 2022

(30) Foreign Application Priority Data
Sep. 23, 2019 (EP) 19199046.4

(51) Int. Cl. C11D 3/386; C12N 9/24; C12N 9/96; C11D 11/00

(52) U.S. Cl. CPC C11D 3/38636; C12N 9/2494; C12Y 302/01078; C12N 9/96; C11D 11/0017

(57) ABSTRACT

Method to remove mannan comprising stains by contacting at least one mannan comprising stain with a mannanase at least 80% identical to SEQ ID NO: 1.

### DESCRIPTION

The main role of hemicelluloses and galactomannans is to function as structural polysaccharide and/or as reserve energy. Besides amylose and amylopectin which are the most widespread storage polysaccharides in plants, there is a diverse group of mannan-based polysaccharides found in seeds, roots, bulbs and tubers of various plants. These include mannans, galactomannans and glucomannans.

Mannans are polysaccharides with a backbone of β-1,4-linked D-mannopyranosyl residues. In most cases the mannans are highly insoluble in water. In contrast to unsubstituted mannans, the galactomannans are water soluble. Due to the complex structural composition of the plant cell wall, microorganisms thriving on decaying plant material must possess a number of different enzymes that are able to hydrolyse these highly polymeric and mostly insoluble materials. The two major endo-acting enzymes involved in degradation of hemicelluloses are beta-mannanase and beta-xylanase. In addition, the exo-acting enzymes beta-mannosidase, alpha-galactosidase and beta-glucosidase are needed for complete degradation of galactoglucomannan.

The main enzyme type participating in the degradation of mannan backbones are endo-1,4-beta-mannanases (EC 3.2.1.78), which hydrolyze the internal glycoside bonds in the mannan backbone. Endo-1,4-β-mannanases (EC 3.2.1.78) are mannan-degrading enzymes which are also called endo-β-1,4-D-mannanase, β-mannanase, or mannanase herein. Since endo-1,4-beta-mannanases (EC 3.2.1.78) degrade the mannan-backbone, mannan-degradation includes the degradation of mannans, galactomannans and/or glucomannans.

The use of mannanase enzymes is widespread in food & feed applications, the detergent and the pulp & paper industry:
The use of mannanase enzymes as feed additives has been shown to provide several beneficial effects since for monogastric animals like poultry and swine mannans are largely indigestible feed components that act as antinutritional factors.
In the food industry mannanase enzymes are described for the use in the production of instant coffee where the enzyme reduces the viscosity of the coffee extracts due to hydrolysis of the coffee mannan. Further, mannanases are used to produce specific mannooligomers that are of interest as functional food ingredients such as mannooligomers with a prebiotic functionality. In such applications plant derived mannopolymers are subjected to hydrolysis by mannanases.
Detergent use: mannanases facilitate the removal of food and cosmetic derived stains/soils that often comprise mannan containing additives like stabilizers, emulsifiers and thickeners. In a more specific cleaning application mannanases are applied to remove biofilms from surfaces or tubings that need to be free from microbials like pharmaceutical equipment. In this application mannanases are often used in combination with detergents and other enzymes like carbohydrases and proteases.
Pulp and paper: mannanases are used in the enzyme-aided bleaching of paper pulp. Mannanases are said to complement the action of xylananses.
Mannanases are applied in the process of oil and gas well stimulation by hydraulic fracturing. Mannanases reduce viscosity of a guar solution that is applied in the process.
Mannanases are used in the controlled release of drugs or other material from matrices that are composed of cross-linked galactomannans.

Activity under application conditions is a critical parameter for many industrially applied enzymes, since these enzymes often tend to be insufficiently active under application conditions.

There is a continuous need for enzymes that perform in the harsh environment of detergent formulations. Different classes of enzymes are known to be useful in detergent formulations such as protease, amylase, cellulase, lipase, mannanase, pectate lyase, and nuclease. Mannanases are useful components of washing and/or cleaning formulations since mannanases remove part of hemicellulose comprising stains. Insufficient removal of these types of stains usually resulting in fabric graying.

Mannan-comprising stains herein comprise at least one mannan, at least one galactomannan and/or at least one glucomannan and in one embodiment, further constituents such as cellulose and/or hemicellulose. Further, such stains may comprise proteinaceous material, starch and/or sugars. Galactomannans usually consist of a mannose backbone with galactose side groups. Herein, galactomannans include galactomannans having the following mannose to galactose ratio: fenugreek gum about 1:1, guar gum about 2:1, tara gum about 3:1, locust bean gum or carob gum about 4:1, cassia gum about 5:1, wherein the ratio is mannose:galactose. Galactomannans are often used in food and cosmetic products to increase the viscosity of a liquid product.

In one embodiment, at least one mannan-comprising stain is comprised on a textile.

Hence, it was the objective to find mannan degrading enzymes catalytically active in formulations having a pH in the range of 5-12, preferably in the range of 6-11, more preferably selected from the ranges of 6-10, 7-9, 7-12, 8-12, 8-10 and 7.5-8.5. Preferably the mannan degrading enzymes show wash performance when provided within a detergent formulation.

In one aspect, the invention provides a mannanase at least 80% identical to SEQ ID NO: 1 provided within an enzyme preparation that allows to be flexibly formulated into liquid detergent formulations or cleaning formulations with either one type of enzymes or mixtures of enzymes. “Formulated into” means that the enzyme preparation is added to a liquid formulation.

In one aspect, the present invention provides a method of removing mannan comprising stains by the steps of contacting at least one mannan comprising stain with a mannanase at least 80% identical to SEQ ID NO: 1. The mannanase has mannan degrading activity at a pH in the range of 5-12 or 6-11, more preferably a pH in the range of 6-10 or 7-9 or 7-12 or 8-12 or 8-10, and most preferably at a pH in the range of 7.5-8.5. At said pH the mannanase shows wash performance on mannan comprising stains. Preferably, the method is a method of removing mannan comprising stains at temperatures 60° C., preferably in the range of about 5-60° C., preferably in the range of about 5-40° C., more preferably in the range of about 10-40° C.

In one aspect, the invention at hand provides a formulation preferably having a pH in the range of 5-12, preferably in the range of 6-11, more preferably in a range selected from 6-10, 7-9, 7-12, 8-12, 8-10 and 7.5-8.5 comprising a mannanase of the invention. Preferably, the formulation is a liquid formulation comprising at least one component selected from surfactants, builders, and hydrotropes is present in amounts effective in maintaining the physical characteristics of the liquid formulation and/or in amounts effective in washing or cleaning. In one embodiment, the formulation is a detergent formulation and the mannanase shows wash performance when provided within the detergent formulation.

Generally, “enzymes” are catalytically active proteins or polypeptides acting on substrates and converting these into products. This reaction is also called enzymatic conversion herein which typically takes place at the “active site” of an enzyme. Enzymes exerting enzymatic conversion are enzymatically active or have enzymatic activity. Any polypeptide called “enzyme” herein means polypeptides being catalytically active.

The mannanases according to the invention have mannan degrading activity and are of the enzyme class EC 3.2.1.78. In one embodiment, mannan degrading activity means degradation of at least one galactomannan. Preferably, at least one galactomannan is characterized by the ratio mannose:galactose of about 1:1, about 2:1, about 3:1, about 4:1, and/or 5:1.

Mannan degrading activity or mannanase activity may be tested according to standard test procedures known in the art. For example: a mannanase to be tested may be applied to 4 mm diameter holes punched out in agar plates comprising 0.2% AZCL galactomannan (carob), i.e. substrate for the assay of endo-1,4-beta-D-mannanase. Carob is e.g. available as I-AZGMA from the company Megazyme. Mannan degrading activity may be tested in a liquid assay using carob galactomannan dyed with Remazol Brilliant Blue as disclosed in McCleary, B. V. (1978); Carbohydrate Research, 67(1), 213-221. Another method to test mannan degrading activity is detection of reducing sugars when incubated with substrate (like guar gum or locust bean gum)—see Miller, G. L. Use of Dinitrosalicylic Acid Reagent for Determination of Reducing Sugars. Analytical Chemistry 1959; 31, 426-428.

Enzymes are polypeptides which are usually identified by polypeptide sequences (also called amino acid sequences herein). Polypeptide sequences are usually identified by a SEQ ID NO. According to the World Intellectual Property Office (WIPO) Standard ST.25 (1998) the amino acids herein are represented using three-letter code with the first letter as a capital or the corresponding one letter.

A “parent” polypeptide amino acid sequence is the starting sequence for introduction of mutations (e.g. by introducing one or more amino acid substitutions, insertions, deletions, or a combination thereof) to the sequence, resulting in “variants” of the parent polypeptide amino acid sequences. A parent includes: A wild-type polypeptide amino acid sequence or synthetically generated polypeptide amino acid sequence that is used as starting sequence for introduction of (further) changes.

The parent polypeptide for the mannanase of this invention has a polypeptide sequence according to SEQ ID NO: 1.

A “variant polypeptide” refers to an enzyme that differs from its parent in its amino acid sequence.

Variant polypeptide sequences in one embodiment is defined by their “sequence identity” when compared to a parent sequence. An enzyme or polypeptide “at least x % identical to SEQ ID NO:X” means an enzyme or polypeptide having a polypeptide sequence which is x % identical when compared to the polypeptide sequence according to SEQ ID NO:X.

Sequence identity usually is provided as “% sequence identity” or “% identity”. For calculation of sequence identities, in a first step a sequence alignment has to be produced.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62).

After aligning two sequences, in a second step, an identity value is determined from the alignment produced.

Herein, the %-identity is calculated by dividing the number of identical residues by the length of the alignment region which is showing the two aligned sequences over their complete length multiplied with 100: %-identity=(identical residues/length of the alignment region which is showing the two aligned sequences over their complete length)*100.

In one embodiment, the mannanase of the invention is at least 80%, at least 85%, at least 90%, at least 91%, at least 92%, at least 93%, at least 94%, at least 95%, at least 96%, at least 97%, at least 98%, or at least 99% identical to SEQ ID NO: 1, preferably when compared to the full length amino acid sequence of SEQ ID NO: 1. The mannanase may further comprise one or more conservative substitutions, meaning that one amino acid is substituted with a similar amino acid. Similar amino acids according to the invention are defined as follows: amino acid A is similar to amino acids S; amino acid D is similar to amino acids E and N; amino acid E is similar to amino acids D, K, and Q; amino acid F is similar to amino acids W and Y; amino acid H is similar to amino acids N and Y; amino acid I is similar to amino acids L, M, and V; amino acid K is similar to amino acids E, Q, and R; amino acid L is similar to amino acids I, M, and V; amino acid M is similar to amino acids I, L, and V; amino acid N is similar to amino acids D, H, and S; amino acid Q is similar to amino acids E, K, and R; amino acid R is similar to amino acids K and Q; amino acid S is similar to amino acids A, N, and T; amino acid T is similar to amino acids S; amino acid V is similar to amino acids I, L, and M; amino acid W is similar to amino acids F and Y; amino acid Y is similar to amino acids F, H, and W.

The mannanase of the invention is a “mature polypeptide” meaning an enzyme in its final form including any post-translational modifications, glycosylation, phosphorylation, truncation, N-terminal modifications, C-terminal modifications, signal sequence deletion. A mature polypeptide can vary depending upon the expression system, vector, promoter, and/or production process.

Enzymes are usually produced as a liquid concentrate, frequently derived from a fermentation broth. “Liquid enzyme concentrate” herein means any liquid enzyme-comprising product comprising at least one enzyme. “Liquid” in the context of enzyme concentrate is related to the physical appearance at 20° C. and 101.3 kPa.

The liquid enzyme concentrate may result from dissolution of solid enzyme in solvent. In such a case, the solvent, preferably is selected from water and an organic solvent. A liquid enzyme concentrate resulting from dissolution of solid enzyme in solvent comprises amounts of enzyme up to the saturation concentration.

Dissolution herein means, that solid compounds are liquified by contact with at least one solvent. Dissolution means complete dissolution of a solid compound until the saturation concentration is achieved in a specified solvent, wherein no phase-separation occurs.

In one aspect of the invention, the enzyme concentrate may be free of water, meaning that no significant amounts of water are present. Non-significant amounts of water herein means, that the enzyme concentrate comprises less than 25%, less than 20%, less than 15%, less than 10%, less than 7%, less than 5%, less than 4%, less than 3%, less than 2% by weight water, all relative to the total weight of the enzyme concentrate, or no water. In one embodiment, enzyme concentrate free of water free of water means that the enzyme concentrate does not comprise significant amounts of water but does comprise organic solvents in amounts of about 10% to 90% by weight, 20% to 85% by weight, 30-80% by weight, 40% to 75% by weight, 50% to 70% by weight, all relative to the total weight of the enzyme concentrate.

Liquid enzyme concentrates comprising water may be called “aqueous enzyme concentrates”. In one embodiment, aqueous enzyme concentrates are enzyme-comprising solutions, wherein solid enzyme product is dissolved in water. In one embodiment “aqueous enzyme concentrate” means enzyme-comprising products resulting from enzyme production by fermentation.

Fermentation means the process of cultivating recombinant cells which express the desired enzyme in a suitable nutrient medium allowing the recombinant host cells to grow and express the desired protein. At the end of the fermentation, fermentation broth usually is collected and further processed, wherein the fermentation broth comprises a liquid fraction and a solid fraction. Depending on whether the enzyme has been secreted into the liquid fraction or not, the desired protein or enzyme is recovered from the liquid fraction of the fermentation broth or from cell lysates. Recovery of the desired enzyme uses methods known to those skilled in the art.

Suitable methods for recovery of proteins or enzymes from fermentation broth include but are not limited to collection, centrifugation, filtration, extraction, and precipitation.

Liquid enzyme concentrates comprise amounts of enzyme in the range of 0.1% to 40% by weight, or 0.5% to 30% by weight, or 1% to 25% by weight, or 3% to 25% by weight, or 5% to 25% by weight, all relative to the total weight of the enzyme concentrate. In one embodiment, liquid enzyme concentrates are resulting from fermentation and are aqueous.

Aqueous enzyme concentrates resulting from fermentation comprise water in amounts of more than about 50% by weight, more than about 60% by weight, more than about 70% by weight, or more than about 80% by weight, all relative to the total weight of the enzyme concentrate. Aqueous enzyme concentrates resulting from fermentation, in one embodiment comprise water in amounts in the range of about 50% to 80% by weight, or about 60% to 70% by weight, all relative to the total weight of the enzyme concentrate. Aqueous enzyme concentrates which result from fermentation, may comprise residual components such as salts originating from the fermentation medium, cell debris originating from the production host cells, metabolites produced by the production host cells during fermentation. In one embodiment, residual components are comprised in liquid enzyme concentrates in amounts less than 30% by weight, less than 20% by weight less, than 10% by weight, or less than 5% by weight, all relative to the total weight of the aqueous enzyme concentrate.

Enzymes tend to lose enzymatic activity if remaining in an aqueous environment and so it is conventional practice to convert it to an anhydrous form: aqueous concentrates may be lyophilized or spray-dried e.g. in the presence of a carrier material to form aggregates. Usually, solid enzyme products need to be “dissolved” prior to use. To stabilize enzymes in liquid products enzyme inhibitors are usually employed, preferably reversible enzyme inhibitors, to inhibit enzyme activity temporarily until the enzyme inhibitor is released.

An enzyme preparation of the invention is preferably liquid. “Liquid” in the context of enzyme preparation is related to the physical appearance at 20° C. and 101.3 kPa.

The enzyme preparation of the invention comprises a liquid enzyme concentrate comprising at least one mannanase of the invention. An enzyme preparation of the invention comprises only components effective in stabilizing the enzyme preparation or the enzyme comprised therein, e.g. selected from at least one enzyme stabilizer, at least one compound stabilizing the liquid enzyme preparation as such, and at least one solvent.

The liquid composition of the invention is preferably free from surfactants. Free from surfactants means, that less than about 10% by weight, less than about 7% by weight, less than about 5% by weight, less than about 3% by weight, less than about 2% by weight, or less than about 1% by weight surfactants are comprised in the liquid composition relative to the total weight of the liquid composition.

The liquid composition of the invention is preferably free from complexing agents. Free from complexing agents means, that less than about 10% by weight, less than about 7% by weight, less than about 5% by weight, less than about 3% by weight, less than about 2% by weight, or less than about 1% by weight complexing agents, preferably aminocarboxylates, are comprised in the liquid composition relative to the total weight of the liquid composition.

In one embodiment, the liquid composition of the invention is free from surfactants and free from complexing agents.

Stabilization of an enzyme relates to stability in the course of time (e.g. storage stability), thermal stability, pH stability, and chemical stability. The term “enzyme stability” herein preferably relates to the retention of enzymatic activity as a function of time e.g. during storage or operation. Enzyme stabilizers stabilize an enzyme in liquid, preferably aqueous environment, meaning that it reduced or avoids loss of enzymatic activity in the course of time.

In one embodiment, at least one enzyme, preferably at least one mannanase of the invention, is stabilized by the presence of water-soluble sources of calcium and/or magnesium ions within the enzyme preparation. In one embodiment, at least one enzyme stabilizer is selected from polyols or water-soluble salts.

Polyols include polyols containing from 2 to 6 hydroxyl groups. Suitable examples include glycol, 1,2-propane diol, 1,2-butane diol, 1,2-pentane diol, ethylene glycol, hexylene glycol, glycerol, sorbitol, mannitol, erythriol, glucose, fructose, and lactose.

Water-soluble salts in one embodiment are selected from salts like NaCl or KCl, and alkali salts of lactic acid and formic acid.

In an embodiment of the invention, at least one water-soluble salt is selected from water-soluble sources of zinc (II), calcium (II) and/or magnesium (II) ions in the finished compositions that provide such ions to the enzymes, as well as other metal ions (e.g. barium (II), scandium (II), iron (II), manganese (II), aluminum (III), Tin (II), cobalt (II), copper (II), Nickel (II), and oxovanadium (IV)). Preferably, the water-soluble salt is selected from CaCl₂ and MgCl₂.

In one aspect of the invention, the enzyme preparation further comprises a protease, preferably a serine protease (EC 3.4.21), more preferably a subtilisin EC 3.4.21.62; and/or a lipase, preferable a triacylglycerol lipase (EC 3.1.1.3), more preferably a Thermomyces lanuginose lipase. An enzyme stabilizer, in this context preferably is selected from boron-containing compounds, polyols, peptide aldehydes, other stabilizers, and mixtures thereof.

Boron-containing compounds are selected from boric acid or its derivatives and from boronic acid or its derivatives such as aryl boronic acids or its derivatives, from salts thereof, and from mixtures thereof. Boric acid herein is also called orthoboric acid.

In one embodiment, at least one boron-containing compound is selected from the group consisting of aryl boronic acids and its derivatives. In one embodiment, boron-containing compound is selected from the group consisting of benzene boronic acid (BBA) which is also called phenyl boronic acid (PBA), derivatives thereof, and mixtures thereof.

In one embodiment phenyl-boronic acid derivatives are selected from the group consisting of 4-formyl phenyl boronic acid (4-FPBA), 4-carboxy phenyl boronic acid (4-CPBA), 4-(hydroxymethyl) phenyl boronic acid (4-HMPBA), and p-tolylboronic acid (p-TBA).

Other suitable derivatives include: 2-thienyl boronic acid, 3-thienyl boronic acid, (2-acetamidophenyl) boronic acid, 2-benzofuranyl boronic acid, 1-naphthyl boronic acid, 2-naphthyl boronic acid, 2-FPBA, 3-FBPA, 1-thianthrenyl boronic acid, 4-dibenzofuran boronic acid, 5-methyl-2-thienyl boronic acid, 1-benzothiophene-2 boronic acid, 2-furanyl boronic acid, 3-furanyl boronic acid, 4,4 biphenyl-diboronic acid, 6-hydroxy-2-naphthaleneboronic acid, 4-(methylthio) phenyl boronic acid, 4-(trimethylsilyl) phenyl boronic acid, 3-bromothiophene boronic acid, 4-methylthiophene boronic acid, 2-naphthyl boronic acid, 5-bromothiophene boronic acid, 5-chlorothiophene boronic acid, dimethylthiophene boronic acid, 2-bromophenyl boronic acid, 3-chlorophenyl boronic acid, 3-methoxy-2-thiophene boronic acid, p-methyl-phenylethyl boronic acid, 2-thianthrenyl boronic acid, di-benzothiophene boronic acid, 9-anthracene boronic acid, 3,5 dichlorophenyl boronic, acid, diphenyl boronic acid anhydride, o-chlorophenyl boronic acid, p-chlorophenyl boronic acid, m-bromophenyl boronic acid, p-bromophenyl boronic acid, p-fluorophenyl boronic acid, octyl boronic acid, 1,3,5 trimethylphenyl boronic acid, 3-chloro-4-fluorophenyl boronic acid, 3-aminophenyl boronic acid, 3,5-bis-(trifluoromethyl) phenyl boronic acid, 2,4 dichlorophenyl boronic acid, 4-methoxyphenyl boronic acid, and mixtures thereof.

In one embodiment, at least one enzyme stabilizer is selected from peptide aldehydes. Peptide aldehydes are selected from di-, tri- or tetrapeptide aldehydes and aldehyde analogues (either of the form B1-BO-R wherein, R is H, CH₃, CX₃, CHX₂, or CH₂X (X=halogen), BO is a single amino acid residue (in one embodiment with an optionally substituted aliphatic or aromatic side chain); and B1 consists of one or more amino acid residues (in one embodiment one, two or three), optionally comprising an N-terminal protection group, or as described in WO 09/118375 and WO 98/13459, or a protease inhibitor of the protein type such as RASI, BASI, WASI (bi-functional alpha-amylase/subtilisin inhibitors of rice, barley and wheat) or Cl₂ or SSI. Preferably, at least one peptide aldehyde is a tripeptide aldehyde.

Compounds stabilizing the liquid enzyme preparation as such means any compound except enzyme stabilizers needed to establish storage stability of a liquid preparation in amounts effective to ensure the storage stability.

Storage stability in the context of liquid preparations to those skilled in the art usually includes aspects of appearance of the product and uniformity of dosage.

Appearance of the product is influenced by the pH of the product and by the presence of compounds such as preservatives, antioxidants, viscosity modifiers, emulsifiers etc.

Uniformity of dosage is usually related to the homogeneity of a product.

Inventive enzyme preparations are alkaline or exhibit a neutral or slightly acidic pH value. The enzyme preparation may have a pH in the range of 5-12 or 6-11, more preferably a pH in the range of 6-10 or 7-9 or 7-12 or 8-12 or 8-10, and most preferably at a pH in the range of 7.5-8.5

In one embodiment, the liquid enzyme preparation of the invention comprises at least one preservative. Preservatives are added in amounts effective in preventing microbial contamination of the liquid enzyme preparation, preferably the aqueous enzyme preparation.

Non-limiting examples of suitable preservatives include (quaternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride(DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L-(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.″-trimethyl-1,3,5-triazine-1,3,5(2H,4H,6H)-triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione.

The enzyme preparation of the invention preferably comprises at least one preservative selected from the group consisting of 2-phenoxyethanol, glutaraldehyde, 2-bromo-2-nitropropane-1,3-diol, and formic acid in acid form or as its salt, and 4,4′-dichloro 2-hydroxydiphenylether. Usually, the enzyme preparation of the invention comprises at least one preservative in amounts ranging from 2 ppm to 5% by weight relative to the total weight of the liquid enzyme preparation. More preferably, the liquid enzyme preparation is free from preservatives, meaning that preservatives are comprised in amounts less than 1 ppm.

In one embodiment, the inventive enzyme preparation is aqueous, comprising water in amounts in the range of 5% to 95% by weight, in the range of 5% to 30% by weight, in the range of 5% to 25% by weight, in the range of 30% to 80% by weight, or in the range of 20% to 70% by weight, all relative to the total weight of the enzyme preparation.

In one embodiment, the enzyme preparation of the invention comprises at least one organic solvent selected from ethanol, n-propanol, iso-propanol, n-butanol, iso-butanol, sec.-butanol, ethylene glycol, propylene glycol, 1,3-propane diol, butane diol, glycerol, diglycol, propyl di-glycol, butyl diglycol, hexylene glycol, ethylene glycol methyl ether, ethylene glycol ethyl ether, ethylene glycol propyl ether, and phenoxyethanol, preferred are ethanol, isopropanol or propylene glycol. Further, the enzyme preparation of the invention may comprise at least one organic solvent selected from compounds such as 2-butoxyethanol, isopropyl alcohol, and d-limonene.

In a preferred embodiment, the enzyme preparation of the invention, comprises at least one water miscible organic solvent. Water miscibility in this context means the property of the organic solvent to mix in all proportions in water, forming a homogeneous solution. Preferably, at least one water miscible solvent is selected from ethanol, isopropanol or 1,2-propylene glycol.

In one embodiment, the enzyme preparation comprises
(a) amounts of water in the range of about 20% to 50% and
(b) at least one organic solvent in amounts in the range of 30% to 60% by weight, or in amounts in the range of 45% to 55% by weight, all relative to the total weight of the enzyme preparation.

In one embodiment, the enzyme preparation comprises organic solvents in amounts in the range of 0% to 20% by weight relative to the total weight of the enzyme preparation. Preferably, the enzyme preparation comprises amounts of water in the range of about 30% to 80% by weight and at least one organic solvent in amounts of less than 10% by weight, less than 5% by weight, or less than 1% by weight, all relative to the total weight of the enzyme preparation.

In one embodiment, the enzyme preparation comprises water in amounts in the range of 5% to 15% by weight and no significant amounts of organic solvent, for example 1% by weight or less, all relative to the total weight of the enzyme preparation.

The present invention provides a method of removing mannan comprising stains by the steps of contacting at least one mannan-comprising stain with a mannanase at least 80% identical to SEQ ID NO: 1. The mannanase has mannan degrading activity at a pH in the range of 5-12 or 6-11, more preferably a pH in the range of 6-10 or 7-9 or 7-12 or 8-12 or 8-10, and most preferably at a pH in the range of 7.5-8.5. At said pH the mannanase shows wash performance on mannan comprising stains.

The mannanase as disclosed herein has mannan degrading activity at a temperature selected from 560° C., 540° C., and 525° C. Therefore, preferably, the method is a method of removing mannan comprising stains at temperatures 560° C., preferably in the range of about 5-60° C., preferably in the range of about 5-40° C., more preferably in the range of about 10-40° C. Preferably, the temperature is washing or cleaning temperature.

In one aspect, the invention relates to a method of removing mannan comprising stains at temperatures 560° C., preferably in the range of about 5-60° C., preferably in the range of about 5-40° C., more preferably in the range of about 10-40° C., by the steps of
(a) providing a liquid formulation comprising at least one mannanase which is at least 80% identical to SEQ ID NO: 1,
(b) contacting a mannan-comprising stain with the liquid formulation of (a)
(c) let the enzyme exert its catalytic activity on the stain for a time in the range of about 10-90 minutes, preferably 20-80 minutes, more preferably 30-70 minutes, or even more preferably 40-60 minutes.

In one embodiment, the method of removing mannan comprising stains is characterized in being a method of washing which is preferably done under mechanical agitation in a laundry machine. The liquid formulation preferably is a liquid detergent formulation.

In one aspect, the invention relates to a formulation preferably having a pH in the range of 5-12 comprising at least one mannanase at least 80% identical to SEQ ID NO: 1, wherein the formulation has increased washing or cleaning performance towards mannan-comprising stains, preferably stains comprising at least one galactomannan, more preferably stains comprising locust bean gum and/or guar gum. Increased washing or cleaning performance towards mannan-comprising stains means increased washing or cleaning performance when compared to a formulation lacking the mannanase of the invention or when compared to a formulation lacking any mannanase.

In one embodiment, the formulation has a pH in the range of 6-11, more preferably in a range selected from 6-10, 7-9, 7-12, 8-12, 8-10 and 7.5-8.5. In one embodiment, the formulation is a detergent formulation, preferably a liquid detergent formulation.

The invention in one aspect relates to the use of the liquid enzyme preparation of the invention to be formulated into detergent formulations such as I&I and homecare formulations for laundry and hard surface cleaning, wherein components (a) and (b) are mixed in no specified order in one or more steps with one or more detergent components. “Formulated into” means that the enzyme preparation is added to a liquid formulation.

The formulation according to the invention comprises one or more detergent component(s). The component(s) chosen depend on the desired washing or cleaning application and/or physical form of the formulation which is also called detergent formulation herein.

The term “detergent component” is defined herein to mean any types of ingredient, which is suitable for detergent formulation, such as surfactants, building agents, polymers, bleaching systems. Any component(s) known in the art acknowledging their known characteristics are suitable detergent component(s) according to the invention. Detergent components in one embodiment means components which provide washing or cleaning performance, or which effectively aid the processing (maintain physical characteristics during processing, storage and use; e.g. rheology modifiers, hydrotropes, desiccants) when present in effective amounts.

Usually, a detergent formulation is a complex formulation of more than two detergent components.

Detergent components may have more than one function in the final application of a detergent formulation, therefore any detergent component mentioned in the context of a specific function herein, may also have another function in the final application of a detergent formulation. The function of a specific detergent component in the final application of a detergent formulation usually depends on its amount within the detergent formulation, i.e. the effective amount of a detergent component.

The term “effective amount” includes amounts of individual components to provide effective stain removal and effective cleaning conditions (e.g. pH, quantity of foaming), amounts of certain components to effectively provide optical benefits (e.g. optical brightening, dye transfer inhibition), and amounts of certain components to effectively aid the processing (maintain physical characteristics during processing, storage and use; e.g. rheology modifiers, hydrotropes, desiccants).

In one embodiment, the detergent formulation according to the invention is a formulation of more than two detergent components, wherein at least one component is effective in stain-removal, at least one component is effective in providing the optimal cleaning conditions, and at least one component is effective in maintaining the physical characteristics of the detergent.

Individual detergent components and usage in detergent formulation are known to those skilled in the art. Suitable detergent components comprise inter alia surfactants, builders, polymers, alkaline, bleaching systems, fluorescent whitening agents, suds suppressors and stabilizers, hydrotropes, and corrosion inhibitors. Further examples are described e.g. in “complete Technology Book on Detergents with Formulations (Detergent Cake, Dishwashing Detergents, Liquid & Paste Detergents, Enzyme Detergents, Cleaning Powder & Spray Dried Washing Powder)”, Engineers India Research Institute (EIRI), 6th edition (2015). Another reference book for those skilled in the art may be “Detergent Formulations Encyclopedia”, Solverchem Publications, 2016.

Detergent components vary in type and/or amount in a detergent formulation depending on the desired application such as laundering white textiles, colored textiles, and wool. The component(s) chosen further depend on physical form of a detergent formulation (liquid, solid, gel, provided in pouches or as a tablet, etc.). The component(s) chosen e.g. for laundering formulations further depend on regional conventions which themselves are related to aspects like washing temperatures used, mechanics of laundry machine (vertical vs. horizontal axis machines), water consumption per wash cycle etc. and geographical characteristics like average hardness of water.

For example: A low detergent concentration system includes laundering formulations where less than about 800 ppm of detergent components are present in the wash water. A medium detergent concentration includes laundering formulations where between about 800 ppm and about 2,000 ppm of detergent components are present in the wash water. A high detergent concentration includes laundering formulations where more than about 2,000 ppm of detergent components are present in the wash water.

The numeric ranges recited for the individual detergent components provide amounts comprised in detergent formulations. Such ranges have to be understood to be inclusive of the numbers defining the range and include each integer within the defined range.

If not described otherwise, “% by weight” or “% w/w” is meant to be related to total detergent formulation. In this case “% by weight” or “% w/w” is calculated as follows: concentration of a substance as the weight of that substance divided by the total weight of the formulation, multiplied by 100.

In one embodiment, the detergent formulation according to the invention comprises one or more surfactant(s). “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric.

Non-limiting examples of surfactants are disclosed McCutcheon's 2016 Detergents and Emulsifiers, and McCutcheon's 2016 Functional Materials, both North American and International Edition, MC Publishing Co, 2016 edition. Further useful examples are disclosed in earlier editions of the same publications which are known to those skilled in the art.

In one embodiment, the detergent according to the invention comprises a total amount of anionic surfactant which in the range of 1% to 30% by weight, in the range of 3% to 25% by weight, in the range of 5% to 20% by weight, or in the range of 8% to 15% by weight, all relative to the total weight of the detergent formulation. In one embodiment, the detergent formulation of the invention comprises a total amount of anionic surfactant of about 11% by weight relative to the total weight of the detergent formulation.

In one embodiment, the detergent composition according to the invention comprises at least one anionic surfactant selected from compounds of general formula (I):

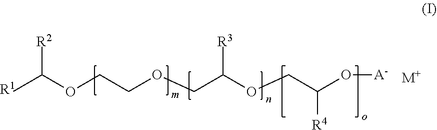

The variables in general formula (I) are defined as follows:

R¹ is selected from C₁-C₂₃-alkyl (such as 1-, 2-, 3-, 4-C₁-C₂₃-alkyl) and C₂-C₂₃-alkenyl, wherein alkyl and/or alkenyl are linear or branched, and wherein 2-, 3-, or 4-alkyl; examples are n-C₇H₁₅, n-C₉H₁₉, n-C₁₃H₂₇, n-C₁₅H₃₁, n-C₁₇H₃₅, i-C₉H₁₉, i-C₁₂H₂₅.

R² is selected from H, C₁-C₂₀-alkyl and C₂-C₂₀-alkenyl, wherein alkyl and/or alkenyl are linear or branched.

R³ and R⁴, each independently selected from C₁-C₁₆-alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

A⁻ is selected from —RCOO⁻, —SO₃⁻ and RSO₃⁻, wherein R is selected from linear or branched C₁-C₈-alkyl, and C₁-C₄ hydroxyalkyl, wherein alkyl is. Compounds might be called (fatty) alcohol/alkyl (ethoxy/ether) sulfates [(F)A(E)S] when A⁻ is SO₃⁻, (fatty) alcohol/alkyl (ethoxy/ether) carboxylate [(F)A(E)C] when A⁻ is —RCOO⁻.

M⁺ is selected from H and salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine. The integers of the general formula (I) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Anionic surfactants of the general formula (I) may be of any structure, block copolymers or random copolymers.

Further suitable anionic surfactants include salts (M⁺) of C₁₂-C₁₈ sulfo fatty acid alkyl esters (such as C₁₂-C₁₈ sulfo fatty acid methyl esters), C₁₀-C₁₈-alkylarylsulfonic acids (such as n-C₁₀-C₁₈-alkylbenzene sulfonic acids) and C₁₀-C₁₈ alkyl alkoxy carboxylates. M⁺ in all cases is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

In one embodiment, the detergent formulation comprises at least two anionic surfactants, selected from compounds of general formula (I), wherein one of said anionic surfactants is characterized in R¹ being C₁₁, R² being H, m being 2, n and o=0, A⁻ being SO₃⁻, M⁺ being Na⁺ and the other surfactant is characterized in R¹ being C₁₃, R² being H, m being 2, n and o=0, A⁻ being SO₃⁻, M⁺ being Na⁺.

In one embodiment, the detergent formulation comprises at least one anionic surfactant selected from compounds of general formula (II):

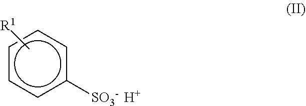

wherein R¹ in formula (II) is C₁₀-C₁₃ alkyl. In one embodiment, the detergent formulation comprises at least two anionic surfactants, selected from compounds of general formula (II), wherein one of said anionic surfactants is characterized in R¹ being C₁₀, and the other surfactant is characterized in R¹ being C₁₃. Compounds like this are also called LAS (linear alkylbenzene sulfonates) herein.

In one embodiment, the detergent formulation of the invention comprises a total amount of non-ionic surfactants in the range of about 1% to about 15% by weight, in the range of about 3% to about 12% by weight, or in the range of about 4% to about 8% by weight, all relative to the total weight of the detergent formulation. In one embodiment, the detergent formulation of the invention comprises a total amount of non-ionic surfactants of about 5.5% by weight relative to the total weight of the detergent formulation.

In one embodiment, the detergent formulation according to the invention comprises at least one non-ionic surfactant according to general formula (III):

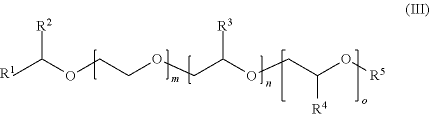

The variables of the general formula (III) are defined as follows:

R¹ is selected from C₁-C₂₃ alkyl and C₂-C₂₃ alkenyl, wherein alkyl and/or alkenyl are linear or branched; examples are n-C₇H₁₅, n-C₉H₁₉, n-C₁₁H₂₃, n-C₁₃H₂₇, n-C₁₇H₃₅, i-C₉H₁₉, i-C₁₂H₂₅.

R² is selected from H, C₁-C₂₀ alkyl and C₂-C₂₀ alkenyl, wherein alkyl and/or alkenyl are linear or branched.

R³ and R⁴, each independently selected from C₁-C₁₆ alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

R⁵ is selected from H and C₁-C₁₈ alkyl, wherein alkyl is linear or branched.

The integers of the general formula (III) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

The non-ionic surfactants of the general formula (III) may be of any structure, is it block or random structure, and is not limited to the displayed sequence of formula (III).

Compounds according to formula (III) are also called alkyl polyethyleneglycol ether (AEO) herein.

In one embodiment, the detergent formulation comprises at least one non-ionic surfactant selected from general formula (III), wherein m is in the range of 3 to 11, preferably not more than 7; n and o is 0, R¹ is C₁₂-C₁₄, R⁵ is H. In one embodiment, the detergent formulation comprises at least two non-ionic surfactants, selected from compounds of general formula (III), wherein one of said non-ionic surfactants is characterized in R¹ being C12, R⁵ being H, m is 7, n and o=0, and the other surfactant is characterized in R¹ being 014, R⁵ being H, m being 7, n and o=0.

The detergent formulation according to the invention, in one embodiment, comprises one or more compounds selected from complexing agents (chelating agents, sequestrating agents), precipitating agents, and ion exchange compounds which may form water-soluble complexes with calcium and magnesium. Such compounds are also called “builders” or “building agents” herein, without meaning to limit such compounds to this function in the final application of a detergent formulation. In one embodiment, the detergent formulation of the invention comprises at least one builder selected from non-phosphate based builders such as sodium gluconate, citrate(s), silicate(s), carbonate(s), phosphonate(s), amino carboxylate(s), polycarboxylate(s), polysulfonate(s), and polyphosphonate(s).

In one embodiment, the detergent formulation of the invention comprises at least one “citrate” selected from the mono- and the dialkali metal salts and in particular the mono- and preferably the trisodium salt of citric acid, ammonium or substituted ammonium salts of citric acid as well as citric acid as such. Citrate can be used as the anhydrous compound or as the hydrate, for example as sodium citrate dihydrate. In one embodiment, the citrate is comprised in a total amount in the range of 0% to about 20% by weight, in the range of about 0.5% to about 10% by weight, or in the range of 1-5% by weight, all relative to the total weight of the detergent formulation. In one embodiment, the detergent formulation of the invention comprises a total amount of citrate in the range of about 1-3% relative to the total weight of the detergent formulation

The detergent formulation of the invention may comprise one or more hydrotropes. In one embodiment, the detergent formulation comprises one or more hydrotropes selected from organic solvents such as ethanol, isopropanol, ethylene glycol, 1,2-propylene glycol, and further organic solvents known in the art that are water-miscible under normal conditions without limitation. In one embodiment, the detergent formulation of the invention comprises 1,2-propylene glycol in a total amount in the range of 5-10% by weight, preferably of about 6% by weight, all relative to the total weight of the detergent formulation.

In one embodiment, the detergent formulation of the invention does not comprise any further enzyme besides the mannanase according to the invention.

In one embodiment, the detergent formulation of the invention comprises at least one further enzyme besides the mannanase of the invention, selected from proteases, amylases, lipases, cellulases, mannanases and any other enzymes known in the art to be useful in detergent formulations.

At least one enzyme which is additionally to the mannanase comprised in the detergent formulation of the invention may itself be stabilized by an enzyme stabilizer. In one aspect, at least one enzyme stabilizer is selected from boron-comprising compounds such as boric acid or its derivatives and boronic acid or its derivatives, from salts thereof, and from mixtures thereof, all as disclosed herein. In one aspect, at least one enzyme stabilizer is selected from peptide aldehydes as disclosed herein.

In one embodiment, at least one enzyme stabilizer is selected from polyols comprising from 2 to 6 hydroxyl groups to stabilize protease. Suitable examples include glycol, 1,2-propane diol, 1,2-butane diol, 1,2 pentane diol, ethylene glycol, hexylene glycol, glycerol, sorbitol, mannitol, erythriol, glucose, fructose, and lactose.

In one embodiment, the detergent formulation of the invention is a laundering detergent.

The term “laundering” relates to both household laundering and industrial laundering and means the process of treating textiles with a solution comprising a detergent formulation of the present invention. In one embodiment, the laundering process is carried out by using technical devices such as a household or an industrial washing machine. A washing machine is also called laundry machine herein. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics (a textile made by weaving, knitting or felting fibers) made of these materials such as garments (any article of clothing made of textile), cloths and other articles.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheep wool, angora, mohair, cashmere). Examples for fibers of synthetic origin are polyurethane fibers such as Spandex® or Lycra®, polyester fibers, polyolefins such as elastofin, or polyamide fibers such as nylon. Fibers means single fibers or parts of textiles such as knitwear, wovens, or nonwovens.

The invention relates to a method to provide a liquid mannanase-comprising formulation, preferably a liquid detergent formulation, more preferably a liquid laundering detergent formulation, comprising the steps of mixing in one or more steps
(a) at least one mannanase at least 80% identical to SEQ ID NO: 1,
   and
(b) at least one detergent component selected from surfactants, builders, and hydrotropes present in amounts effective in cleaning and/or effective in maintaining the physical characteristics of the detergent.

In one embodiment, the invention relates to a method to provide a liquid mannanase-comprising formulation, comprising the steps of mixing in one or more steps in any order
(a) the enzyme preparation of the invention comprising at least one mannanase at least 80% identical to SEQ ID NO: 1,
   and
(b) at least one detergent component selected from surfactants, builders, and hydrotropes present in amounts effective in cleaning and/or effective in maintaining the physical characteristics of the liquid formulation.

In one embodiment, the formulation has a pH in the range of 5-12 or 6-11, more preferably in a range selected from 6-10, 7-9, 7-12, 8-12, 8-10 and 7.5-8.5. In one embodiment, the formulation is a detergent formulation, preferably a liquid detergent formulation, more preferably a liquid laundering detergent formulation.

The laundering detergent formulation of the invention exerts wash performance which is evaluated under relevant wash conditions. The term “relevant wash/cleaning conditions” herein refers to the conditions, particularly temperature, time, cleaning mechanics, suds concentration, type of detergent and water hardness, actually used in laundry machines, or in manual washing processes. In one embodiment, wash performance herein is related towards removal of mannan-comprising stains; preferably mannan-comprising stains are selected from those comprising galactomannans and glucomannans. In one embodiment, wash performance relates to removal of stains comprising galactomannan, more preferably locust bean gum and/or guar gum.

The invention relates to the use of at least one mannanase at least 80% identical to SEQ ID NO: 1 to increase washing or cleaning performance of a detergent formulation towards mannan-comprising stains, preferably stains comprising at least one galactomannan, more preferably stains comprising locust bean gum and/or guar gum.

In one embodiment, the detergent formulation has a pH in the range of 5-12 or 6-11, more preferably in a range selected from 6-10, 7-9, 7-12, 8-12, 8-10 and 7.5-8.5. In one embodiment, the formulation is a liquid detergent formulation, preferably a liquid laundering detergent formulation.

In one embodiment, the washing or cleaning performance is increased at washing or cleaning temperatures 60° C., preferably in the range of about 5-60° C., preferably in the range of about 5-40° C., more preferably in the range of about 10-40° C.

In one aspect, the invention relates to a method of washing or cleaning, comprising the steps of
(a) providing at least one mannan-comprising stain;
(b) providing a detergent formulation according to the invention
(c) contacting the mannan-comprising stain with the detergent of (b).

Preferably, the mannan-comprising stain comprises at least one galactomannan, more preferably locust bean gum and/or guar gum.

In one embodiment, in step (a) a textile comprising mannan-comprising stains is provided.

In one embodiment, the mannanase comprised in the detergent according to the invention removes the mannan-comprising stains from the textile (a) by (c) contacting the mannan-comprising stain with a detergent of (b).

The invention includes the following embodiments:
1 Method of removing mannan comprising stains by the steps of contacting at least one mannan comprising stain with a mannanase at least 80% identical to SEQ ID NO: 1.
2. A liquid enzyme preparation comprising a mannanase at least 80% identical to SEQ ID NO: 1, at least one compound stabilizing the liquid enzyme preparation as such, at least one solvent, and optionally at least one enzyme stabilizer.
3. The enzyme preparation according to embodiment 2 having a pH in the range of 5-12, wherein the enzyme preparation is preferably liquid.
4. A formulation with increased washing or cleaning performance towards mannan-comprising stains, preferably towards stains comprising at least one galactomannan, preferably locust bean gum and/or guar gum, wherein the formulation comprises a mannanase at least 80% identical to SEQ ID NO: 1.
5. The formulation according to embodiment 3, wherein the formulation has a pH in the range of 5-12, preferably wherein the formulation is liquid.
6. The formulation according to embodiments 4 and 5, wherein the formulation comprises at least one component selected from surfactants, builders, and hydrotropes in amounts effective in maintaining the physical characteristics of the formulation and/or in amounts effective in cleaning.
7. The formulation according to embodiments 4-6, wherein the formulation preferably is a detergent formulation, more preferably a laundering detergent formulation, more preferably a liquid laundering detergent.
8. A method to provide a liquid detergent effective towards mannan comprising stains, comprising the steps of mixing in one or more steps
   (a) at least one mannanase at least 80% identical to SEQ ID NO: 1, preferably wherein the mannanase is provided within an enzyme preparation according to embodiment 2 and 3
      and
   (b) at least one component selected from surfactants, builders, and hydrotropes present in amounts effective in cleaning and/or effective in maintaining the physical characteristics of the detergent.
9. A method of washing or cleaning, comprising the steps of
   (a) providing at least one mannan-comprising stain, preferably wherein the mannan-comprising stain comprises at least one galactomannan, more preferably wherein the mannan-comprising stain comprises locust bean gum and/or guar gum;
   (b) providing a formulation according to embodiments 4-6
   (c) contacting the mannan-comprising stain (a) with the detergent of (b), preferably at a washing or cleaning temperature in the range of 5-60° C.
10. The method according to embodiment 8, wherein the mannan-comprising stain is comprised on a textile (a); and wherein the polypeptide comprised in the detergent (b) removes the mannan-comprising stain from the textile (a).
11. Use of at least one mannanase at least 80% identical to SEQ ID NO: 1 to increase wash performance of a detergent formulation towards mannan-comprising stains, preferably at washing or cleaning temperatures in the range of 5-60° C.

### EXAMPLE 1

### Evaluation of Mannanases in Rotawash

The following mannanase enzymes Man01, Man02, and Man03 were tested in liquid detergent ES1_C (pH 8) and Persil non-Bio (pH 7.2) at 40° C. or 25° C. washing temperature:

Man01: mannanase according to SEQ ID NO:1

Man02: Mannanase according to SEQ ID NO:2

Man03: Mannanase according to SEQ ID NO:3

| ES1_C | | | |
|---|---|---|---|
| components | | Conc. | wt % |
| Maranil DBS/LC | linear alkylbenzene | | 5.5 |
| | sulfonate, anionic | | |
| | surfactant | | |
| Edenor coco fatty | C₁₂-C₁₈ | | 2.4 |
| acid | coco fatty acid | | |
| Lutensol AO7 | alkyl polyethyleneglycol | | 5.5 |
| | ether, non-ionic | | |
| | surfactant | | |
| Texapon N70 | sodium laureth | | 5.5 |
| | sulfate 2EO, | | |
| | anionic surfactant | | |
| 1,2 propylene | | | 6.0 |
| glycol | | | |
| C₂H₅OH | | | 2.0 |
| KOH | | | 2.2 |
| Citrate | | | 3.0 |
| Mannanase | | according to Tables | |
| | | Ex1a and Ex1b | |
| pH | | | 8-8.5 |
| H₂O | up to 100% | | |

CFT C-S-43/guar gum stain monitors or CFT C-S-73/locust bean gum stain monitors (CFT, Vlaardingen, NL) were washed together with cotton ballast fabric and steel balls in wash liquor using base formulation ES1_C in the Rotawash (Rotawash M228, SDL Atlas Inc., USA) under the following washing conditions:

| Washing liquor | 500 | ml |
|---|---|---|
| # Steel balls | 20 | |
| Washing time/temperature | 40 min at 25° C. or 40° C. | |
| Dosage Mannanase | From 0 to 0.1 ppm | |
| Dosage detergent | 1 g/L ES1_C or 3.5 g/L Persil non-Bio | |
| Washing cycles | 1 | |
| Water hardness | 2.7 | mmol/L |

| | |
|---|---|
| Ballast fabric | 4 g cotton fabric 283 |
| Soiled swatches | 2 × 0.4 g CS-43 or |
| | 2 × 0.4 g C-S-73 |

After the washing, the fabrics were rinsed and dried in the air. The wash performance for the single stains was determined by measuring the average color intensity of the soiled fabric after wash with a flatbed color image scanner from EPSON (Expression 11000XL). In general, the higher the average intensity value, the better the performance. The results are also outlined below in Tables Ex1a and Ex1b and Ex1c.

TABLE Ex1a
Man01 wash performance at 40° C. on guar
gum stain (C-S-43) in ES1_C detergent formulation;
values in average intensity (RGB)

| Dose | values in average intensity (RGB) | | |
|---|---|---|---|
| ppm | SEQ ID NO: 2 | Man01 | SEQ ID NO: 3 |
| 0 | 0.64 | 0.64 | 0.64 |
| 0.01 | 0.67 | 0.68 | |
| 0.02 | 0.68 | 0.68 | 0.67 |
| 0.05 | 0.68 | 0.70 | |
| 0.1 | 0.68 | 0.72 | 0.69 |

TABLE Ex1b
Man01 wash performance at 40° C. on locust bean
gum stain (C-S-73) in ES1_C detergent formulation;
values in average intensity (RGB)

| Dose | values in average intensity (RGB) | | |
|---|---|---|---|
| ppm | SEQ ID NO: 2 | Man01 | SEQ ID NO: 3 |
| 0 | 0.70 | 0.70 | 0.70 |
| 0.05 | 0.78 | 0.78 | |
| 0.1 | 0.76 | 0.78 | 0.80 |

TABLE Ex1c
Man01 wash performance at 40° C. on guar gum stain (C-
S-43) in Persil non-Bio detergent; values average intensity (RGB)

| Dose | values in average intensity (RGB) | | |
|---|---|---|---|
| ppm | SEQ ID NO: 2 | Man01 | SEQ ID NO: 3 |
| 0 | 0.57 | 0.57 | 0.57 |
| 0.01 | 0.60 | 0.64 | 0.60 |
| 0.05 | 0.62 | 0.66 | 0.64 |
| 0.1 | 0.64 | 0.66 | 0.65 |

TABLE Ex1d
Man01 wash performance at 40° C. on locust bean gum stain
(C-S-73) in Persil non-Bio detergent; values average intensity (RGB)

| Dose | values in average intensity (RGB) | | |
|---|---|---|---|
| ppm | SEQ ID NO: 2 | Man01 | SEQ ID NO: 3 |
| 0 | 0.64 | 0.64 | 0.64 |
| 0.01 | 0.74 | 0.74 | 0.70 |
| 0.05 | 0.73 | 0.73 | 0.74 |
| 0.1 | 0.73 | 0.73 | 0.74 |

TABLE Ex1e
Man01 wash performance at 25° C. on guar gum stain (C-S-
43) in Persil non-Bio detergent; values in average intensity (RGB)

| Dose | values in average intensity (RGB) | | |
|---|---|---|---|
| ppm | SEQ ID NO: 2 | Man01 | SEQ ID NO: 3 |
| 0 | 0.58 | 0.58 | 0.58 |
| 0.01 | 0.60 | 0.64 | |
| 0.02 | 0.61 | 0.64 | 0.61 |
| 0.05 | 0.62 | 0.67 | |
| 0.1 | 0.63 | 0.68 | 0.65 |

1. Method of removing mannan comprising stains at temperatures in the range of about 5-60° C. by the steps of contacting at least one mannan comprising stain with a mannanase at least 80% identical to SEQ ID NO: 1.

2. Method according to claim 1, wherein the mannan-comprising stain is comprised on a textile.

3. A liquid enzyme preparation comprising a mannanase at least 80% identical to SEQ ID NO: 1, at least one compound stabilizing the liquid enzyme preparation as such, at least one solvent, and optionally at least one enzyme stabilizer.

4. The liquid enzyme preparation according to claim 2, wherein the enzyme preparation has a pH in the range of 5-12.

5. A laundering detergent formulation with increased washing or cleaning performance towards mannan-comprising stains, wherein the formulation comprises a mannanase at least 80% identical to SEQ ID NO: 1, and wherein the formulation has a pH in the range of 5-12.

6. The formulation according to claim 5, wherein the formulation comprises at least one component selected from surfactants, builders, and hydrotropes in amounts effective in maintaining the physical characteristics of the formulation and/or in amounts effective in cleaning.

7. A method to provide a liquid laundering detergent effective towards mannan comprising stains, comprising the steps of mixing in one or more steps
(a) at least one mannanase at least 80% identical to SEQ ID NO: 1, and
(b) at least one detergent component selected from surfactants, builders, and hydrotropes present in amounts effective in cleaning and/or in amounts effective in maintaining the physical characteristics of the detergent.

8. A method of washing or cleaning, comprising the steps of
(a) providing at least one mannan-comprising stain;
(b) providing a formulation according to claim 5
(c) contacting the mannan-comprising stain (a) with the formulation of (b).

9. The method according to claim 8, wherein the mannan-comprising stain is provided on a textile (a); and wherein the polypeptide comprised in the detergent (b) removes the mannan-comprising stain from the textile (a).

10. The method according to claim 9, wherein the mannan-comprising stain comprises at least one galactomannan.

11. (canceled)

12. The method of claim 1, wherein the mannan comprising stain comprises at least one galactomannan.

13. The laundering detergent formulation of claim 5 with increased washing or cleaning performance towards mannan-comprising stains comprising locust bean gum and/or guar gum.

14. The laundering detergent formulation of claim 5, wherein the formulation is liquid.

15. The method of washing or cleaning of claim 8, wherein the mannan-comprising stain is contacted with the formulation of (b) at a washing or cleaning temperature in the range of 5-60° C.

16. The method according to claim 10, wherein the mannan-comprising stain comprises locust bean gum and/or guar gum.
